(19) United States
(12) Patent Application Publication
(10) Pub. No.: US 2003/0219464 A1
(43) Pub. Date: Nov. 27, 2003

(54) Method and means for skin rejuvenation including hair growth stimulation

(76) Inventors: Peter Anthony Incando, Monrovia, CA (US); Hitoshi Tamaribuchi, Elk Grove, CA (US)

Correspondence Address: JAMES T. ENGLISH, 1134 Calle Adra St., Duarte, CA, 91010, US

(21) Appl. No.: 10/152,499

(22) Filed: May 23, 2002

(51) Int. Cl. A61K 7/06; A61K 7/00

(52) U.S. Cl. 424/401; 424/74

(57) ABSTRACT

A mixture of substances demonstrated to have a regenerating effect in the treatment of skin and hair abnormalities is based on a treatment method for application of a deep cleanser, a defoliator and stimulator products, together with the method steps for their application that can be accomplished routinely by anyone without professional aesthetician services after initial recommendations are made for treating normal degenerative conditions of the skin and hair. Specific product compositions and recommended methods for their application for exfoliation, cleansing and stimulation are given for routine personal health and beauty treatment use.

### FIELD OF INVENTION

The invention relates to enhancing the health and appearance of the skin, and the skin in which hair grows, particularly after deleterious disease and medical procedures that deplete the chemical balances that are required for normal heathy skin, and hair growth. Methods for skin rejuvenation are classified in Class 514, subclass 884.

It is well known that certain naturally occurring substances and derivatives thereof can be used to enhance the growth of Collagen which is the basic structural material of the dermis and which, when healthy, gives the outer skin a rounded, and smooth youthful appearance. The elimination of microscopic bacteria is also a forward step in producing healthy, youthful skin. Research into the subject has uncovered many materials, both natural and synthetic, that in some degree produce an improved appearance of the skin but the prior art has not produced an easy routine treatment to remove scaling, clean pores, and produce healthy stimulation of hair roots and achieve long lasting changes in the appearances of facial skin.

### DESCRIPTION OF THE PRIOR ART

The prior art teaches the use of Derm abrasion as a technique to bring new-skin tissue to the epidermis, using specialized equipment in the hands of skilled aestheticians in a clinical environment. Pumice is used as the abrasive, and the direction of application, as well as the pressure and motion are all important in bringing a new surface to the epidermis. Other treatments to bring forth new skin consist of counter-irritants in liquid or cremes for exfoliating the epidermis when Acne, scaling, sun burning, and other sebaceous disorders are present.

To maintain the new skin appearance, in the presence of daily assault by the sun, the makeup routines, perspiration, and in some cases actual disease such as Acne and Cancer for which radiation treatment is used, only Moisturizers and Scrubs including vitamins are available. Suction devices have been used to assist in cleaning the pores.

### BRIEF DESCRIPTION OF THE INVENTION

For a dermis rejuvenation that can be performed routinely, the invention provides the new materials and procedures therefor. The new procedures and materials for removing aged , mutilated, sunburned, and medical procedures aggravated skin, a method and materials for exfoliating old skin and stimulating and nourishing the growth of new skin and hair follicles uses proprietary solutions held in a brush that is vibrated or stroked under the user's direction or applied manually to the area to be exfoliated, or stimulated as with hair follicles. After exfoliation, a procedure to re-energize hair follicles involves brushing-on the proprietary Glycolic acid/hydroxypropyl methyl cellulose solution in accordance with the invention. It is intended that the rejuvenation routines can be performed without clinical or salon assistance after diagnosis of the condition.

### DETAILED DESCRIPTION OF THE INVENTION

The novelty of the invention is based on the new combination of materials that compose the products applied sequentially for skin rejuvenation. The FIRST STEP of the routine is to apply the cleanser consisting of the ingredients in Table I and applying organic virgin honey to deeply cleanse, exfoliate, and increase cell renewal while smoothing and protecting the skin; and delivering nutrients deep into the inner dermis, hydrating, rejuvenating and soothing the skin and absorbing impacted cell debris that block pores diminishing pore size; thus imparting clarity, elasticity and tone to the skin surface. The SECOND STEP of the skin rejuvenation routine is applying the novel dermabrasion compound soap having 0.5 percent to 90 percent floured Pumice in a Glycerin base with honey 40 percent of ingredients. The soap can be stick, molded bar, rounded, facial curvature, or pad, with the method for use being repetitious stroking with very light pressure, rubbed generally along wrinkles or non-rounded features.

TABLE I

| | |
|---|---|
| Item | Percentage |

| | |
|---|---|
| Deionized water | 22 |
| Glycerin | 21 |
| Pure virgin Avocado Honey | 20 |
| Sodium Laurel Sulfate (Cocoanut oil) | 9 |
| Stearic Acid | 5.5 |
| Lemon peel | 4 |
| Potassium Hydroxide | 5 |
| Sodium Sulfate | 3 |
| floured Pumice | 2.25 |
| Methyl Paraben | 2 |
| Lanolin | 1.5 |
| Linoleic Acid | 1 |
| Oleic Acid | 1 |
| Vitamin A | .75 |
| Vitamin D-3 | .75 |
| Vitamin E | .75 |
| Biotin | .50 |
| FDC yellow No. 5 | |

For stimulating hair growth, a procedure in accordance with the present skin rejuvenation invention is done as follows preferably after the dermis is cleaned and examined with a microscope to determine the presence of viable hair follicles followed by shampooing with Glycolic Gel and after 5 minutes, brushing and after applying Herbs #1 for 5 minutes, rinsing with tepid water, and applying Facial Glycolic MD with a brush in half-inch sections of hair and letting set for 30 minutes—not to exceed 1 hour; then mixing approximately 2 to 3 ounces of Hair Growth Cleansing Treatment added to Herbs #1 to make a cream mixture. Then dipping Derma Bar into the cream mixture and rubbing in one direction on the section of the hair. This is repeated for all sections of the hair. This is done in both directions until the entire head is treated and the material left on approximately 10 minutes, rinsed and towel dried Herbs #1 can be put on the eyebrows and frontal hair line and left in. Herbs #2 is then sprayed onto the hair which is then blow dried.

HERBS #2

Fari
Angelica Root
Angelica Dahur
Reh Mania Root
Solomonseal
Luqusticum
Cloves
Citri-Medic
Cardamon
Distilled Water

What is claimed is:

1. The method for hair follicle analysis and treatment which comprises:
Examining skin epidermis microscopically for observation of presence of live hair follicles or the absence thereof, to determine if stimulation treatment would be beneficial;
If deemed beneficial, ˜Shampooing the skin containing the hair follicles with Glycolic Gel letting set 3-5 minutes;
Separating hairs for treatment in rows and areas and Brushing and Rinsing area well;
Applying Herbs #1 to the scalp and allowing to set and rinsing with tepid water;
Applying Glycolic GEL. and scrubbing with brush in half-inch sections;
Allowing to set at least 30 minutes not exceeding one hour;
Mixing 2 to 3 ounces of Hair Growth and Cleansing Treatment and adding Herb #1 making a cream mixture;
Dipping Derma Bar into said cream mix, sectioning, and rubbing in one direction;
Brushing the same section up and down until entire area is treated, allowing to stand at least ten minutes more and rinsing and drying;
Spraying Herbs #1 on eyebrows and on entire area of dermis being treated;
Spraying herbs #2 onto the hair and applying bond into palm of hand, and applying to hair and dermis.
Combing; styling, and blow drying the hair and dermis.

2. The composition for hair follicle stimulation which comprises Herbs #1
HERBS #1
TNT HERBS.
Fari
Angelica root
Angelica Dahur
Reh Mania Root
Solomonseal
Liqusticum
Cloves
Citri-Medic
Cardamon
Distilled Water

3. The composition for hair growth dermal cleansing which comprises:
HAIR GROWTH CLEANSING TREATMENT
Ingredients:
Glycerin
pure Honey
pure Cocoanut oil
sodium cocoale
cocamidolopropyl
Hydroxy sultaine
propylene/glycol USP
powdered flower pumice
Tee Laurel sulfate
Sorbitol
Acetamide Mea
Sodium Laurel sulfate
Organic lemon peel powder
lanolin
aloe vera
Methylparaben
sodium Chloride
Sodium sulfate
Potassium Hydroxide
Stearic acid
F.D. and yellow
Natural vitamins A, D, E, & organic
Chinese herbs (made in USA)

4. The composition for hair beauty enhancement, which comprises:
HERBS #2
Cocamidopropyl
Sulfine propyl glycol
USP powdered flour pumice (T80)
Laurel Sulfate
PAS: powder
Lanolin
Aloe
Potassium hydroxide
Stearic acid
F.D. yellow #5

5. A derma-brasion compound soap having 0.5 percent to 99 percent floured Pumice in a Glycerine base with honey 40 percent of ingredients.

6. The soap of claim 5 in a saturated flexible pad.

7. The soap of claim 5 in a facial feature conforming molded bar.

8. The method for skin rejuvenation, which comprises:
Applying the facial cleanser consisting of the ingredients in claim 3
Applying derma-brasion compound soap heaving 0.5 percent to 90 percent floured Pumice in a Glycerine base with Honey
Repetitiously stroking skin with said soap with very light pressure, generally along wrinkles and non-rounded features; and assisting growth of new skin with the vitamins C, D and E;

9. The method of claim 8 further comprising the step of vacuum cleaning the skin after treatment to remove any sebaceous abnormalities and residue.

10. The method of claim 9 further comprising vacuuming along wrinkles for rounding skin depressions by collagen redistribution substitution therefor.
